(19) United States
(12) Patent Application Publication
(10) Pub. No.: US 2025/0318715 A1
(43) Pub. Date: Oct. 16, 2025

(54) AUXILIARY CONTROL SYSTEMS FOR ENDOSCOPE DEVICES

(71) Applicant: Loma Linda University, Lomda Linda, CA (US)

(72) Inventor: Gautam Nagendra, Loma Linda, CA (US)

(21) Appl. No.: 19/177,325

(22) Filed: Apr. 11, 2025

Related U.S. Application Data
(60) Provisional application No. 63/633,028, filed on Apr. 11, 2024.

(51) Int. Cl. A61B 1/005; A61B 1/00

(52) U.S. Cl. CPC A61B 1/0052; A61B 1/00042

(57) ABSTRACT

Disclosed herein is an endoscope system. The endoscope system includes an insertion tube comprising a bendable tube segment at a distal end of the insertion tube, a body comprising a plurality of angulation control knobs configured to control positioning of the bendable tube segment of the insertion tube, and a control system comprising an input device, a knob motor positioned proximate to each angulation control knob, and a knob engagement disc releasably secured to each angulation control knob. The input device is communicatively coupled to each knob motor such that movement of the input device activates the knob motors and controls movement of the angulation control knobs via the knob engagement discs. The positioning of the input device facilitates improving one or more ergonomic conditions of a hand grip of an operator of the endoscope system.

### RELATED APPLICATIONS

The present disclosure claims priority to U.S. Provisional Application No. 63/633,028 entitled JOYSTICK CONTROL FOR ENDOSCOPE DEVICE and filed on Apr. 11, 2024; the entire contents of which are hereby incorporated by reference.

### FIELD

The present disclosure relates to endoscopes for use in medical procedures. In particular, the present disclosure relates to an endoscope device with an auxiliary control system that may be configured as a joystick or one or more sliders that are positioned for improved ergonomic operation.

### BACKGROUND

Endoscopes are devices that contain a forward viewing camera at the end of a flexible tube and are used in multiple fields for minimally invasive procedures. Many endoscopy procedures utilize a reusable endoscope device including a plurality of valve cylinders and a control section that includes a plurality of dials. The dials are generally rotated with a thumb of an operator to control deflection of a distal end of the flexible tube, while one or more fingers of the operator are used to control valves inserted into the valve cylinders.

Many endoscopy procedures may require operators to exert high pinch forces, perform repetitive hand motions, endure contact stress, and/or maintain awkward postures while operating the endoscope. This may increase an operator's risk of sustaining work-related musculoskeletal injuries, including, but not limited to, pain in the thumb, neck, back, and/or shoulder(s). Furthermore, the risk of sustaining injury may be increased for an operator with a smaller hand size or with existing musculoskeletal disease (e.g., arthritis). Thus, there is a need for a control system configured as a joystick or as one or more sliders that is positioned for improved ergonomic operation of the control knobs of an endoscope device.

### SUMMARY

Some aspects of the present disclosure provide an endoscope system and methods of use thereof. These aspects include devices and methods of use. According to one aspect, an endoscope system includes an insertion tube comprising a bendable tube segment at a distal end of the insertion tube, a body comprising a plurality of angulation control knobs configured to control positioning of the bendable tube segment of the insertion tube and a control system comprising an input device, a knob motor positioned proximate to each angulation control knob, and a knob engagement disc releasably secured to each angulation control knob. The input device is communicatively coupled to each knob motor such that movement of the input device activates the knob motors and controls movement of the angulation control knobs via the knob engagement discs and the positioning of the input device facilitates improving one or more ergonomic conditions of a hand grip of an operator of the endoscope system.

In other aspects, the plurality of angulation control knobs comprise a first angulation control knob configured to control positioning along a first bending axis and a second angulation control knob configured to control positioning along a second bending axis. Additionally, a first knob motor is positioned proximate to the first angulation control knob and a second knob motor is positioned proximate to the second angulation control knob.

According to various aspects, the input device is a joystick and wherein the when the joystick is moved along a first axis to control positioning of the bendable tube segment of the insertion tube along the first bending axis and the joystick is moved along a second axis to control positioning of the bendable tube segment of the insertion tube along the second bending axis. The first axis is substantially parallel to the first bending axis in a left-right orientation and the second axis is substantially parallel to the second bending axis in an up-down orientation. The knob motors comprise one or more gears to engage with one or more protrusions around an exterior circumferential surface of one of the knob engagement discs.

According to various other aspects, the input device comprises a slider, wherein moving the slider along an axis in a first direction causes the bendable tube segment of the insertion tube to bend along a bending axis in the first direction and wherein moving the slider in a second direction along the axis causes the bendable tube segment of the insertion tube to bend along the first bending axis opposite the first direction. Additional, the input device comprises another slider and wherein moving the other slider along in the first direction causes the bendable tube segment of the insertion tube to bend along a second bending axis in a second direction and wherein moving the slider in the second direction along the axis causes the bendable tube segment of the insertion tube to bend along the second bending axis opposite the first direction.

In various aspects, the speed of the slider along an axis in a first direction correlates to a speed of deflection for the bendable tube segment. Similarly, a distance of the slider from a neutral position corresponds to a deflection for the bendable tube segment. Additionally, depressing the slider causes the bendable tube segment to return to a neutral position.

In other aspects, the input device comprises two sliders and wherein depressing a first slider causes the bendable tube segment to return to a neutral position along a first axis and depressing a second slider causes the bendable tube segment to return to a neutral position along a second axis. In some aspects, the input device and the knob motors are releasably secured to the body and/or the body includes a panel to cover at least a portion of the control system wherein the panel covers the knob motors. The control system may be detachable from the body and the insertion tube. Alternatively, the control system is integrated with the body.

According to one aspect, a method of using an endoscope system is disclosed. The endoscope system comprises an insertion tube comprising a bendable tube segment at a distal end of the insertion tube, a body comprising a plurality of angulation control knobs configured to control positioning of the bendable tube segment of the insertion tube, and a control system comprising a, input device, a knob motor positioned proximate to each angulation control knob, and a knob engagement disc releasably secured to each angulation control knob, wherein the input device is communicatively coupled to each knob motor such that movement of the input device activates the knob motors and controls movement of the angulation control knobs via the knob engagement discs. The method includes inserting the distal end of the insertion tube into a patient, advancing the distal end of the insertion tube to a desired location within the patient, operating the endoscope system, wherein positioning of the input device facilitates improving one or more ergonomic conditions of a hand grip of an operator of the endoscope system, and removing the insertion tube from the patient.

According other aspects of the disclosed methods the plurality of angulation control knobs comprise a first angulation control knob configured to control positioning along a first bending axis and a second angulation control knob configured to control positioning along a second bending axis, wherein a first knob motor is positioned proximate to the first angulation control knob and a second knob motor is positioned proximate to the second angulation control knob. In various aspects, the input device is a joystick and when the joystick is moved along a first axis to control positioning of the bendable tube segment of the insertion tube along the a first bending axis and the joystick is moved along a second axis to control positioning of the bendable tube segment of the insertion tube along the second bending axis. In another aspect, the input device comprises one or more sliders and wherein when a first slider is moved along a first axis to control positioning of the bendable tube segment of the insertion tube along a first bending axis and a second slider is moved along a second axis to control positioning of the bendable tube segment of the insertion tube along a second bending axis.

According to some aspects, depressing the first slider causes the bendable tube segment of the insertion tube to return to a neutral position along the first bending axis or depressing the second slider causes the bendable tube segment of the insertion tube to return to the neutral position along the second bending axis. The knob motors comprise one or more gears to engage with one or more protrusions around an exterior circumferential surface of one of the knob engagement discs. In numerous aspects, the one or more ergonomic conditions comprise at least one of a force exertion, a contact stress, and a posture. The hand grip of the operator includes a thumb positioned to move the input device.

### BRIEF DESCRIPTION OF THE DRAWINGS

The description will be more fully understood with reference to the following figures and data graphs, which are presented as various embodiments of the disclosure and should not be construed as a complete recitation of the scope of the disclosure. It is noted that, for purposes of illustrative clarity, certain elements in various drawings may not be drawn to scale. Understanding that these drawings depict only exemplary embodiments of the disclosure and are not therefore to be considered to be limiting of its scope, the principles herein are described and explained with additional specificity and detail through the use of the accompanying drawings in which:

FIGS. 1A and 1B are perspective views of an endoscope device as known in the prior art.

FIGS. 2A and 2B are perspective views of an operator using an endoscope device as known in the prior art.

FIG. 2C is a side view of an example difference in hand size.

FIG. 2D is a perspective view of an operator with a smaller hand size using an endoscope device as known in the prior art.

FIG. 3 is a schematic of an endoscope device with a control system including a joystick that is positioned for improved ergonomic operation according to one or more embodiments.

FIG. 4 is a schematic of the endoscope device shown in FIG. 3 including a panel over at least a portion of the control system according to one or more embodiments.

FIG. 5 is a schematic of an endoscope device with a control system including one or more sliders positioned for improved ergonomic operation according to one or more embodiments.

FIG. 6 is a schematic of the endoscope device shown in FIG. 5 including a panel over at least a portion of the control system according to one or more embodiments.

Reference characters indicate corresponding elements among the views of the drawings. The headings used in the figures do not limit the scope of the claims.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and such references mean at least one of the embodiments.

Reference to “one embodiment”, “an embodiment”, or “an aspect” means that a particular feature, structure, or characteristic described in connection with the embodiment is included in at least one embodiment of the disclosure. The appearances of the phrase “in one embodiment” or “in one aspect” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein.

According to various aspects, the auxiliary control systems for endoscopic devices are universally compatible with a wide variety of existing endoscopes and colonoscopes. As such, the devices of the present disclosure may eliminate or minimize the need for costly new scope purchases thus preserving prior investments in equipment, while also reducing adoption barriers by retrofitting existing equipment.

In particular, the adoption barriers are lowered as the various features of the auxiliary control systems are positioned at a natural thumb rest position. Use of the auxiliary system maintains familiar hand positioning near original dial location for users, preserves established procedural muscle memory, and enables a seamless transition from manual to powered control.

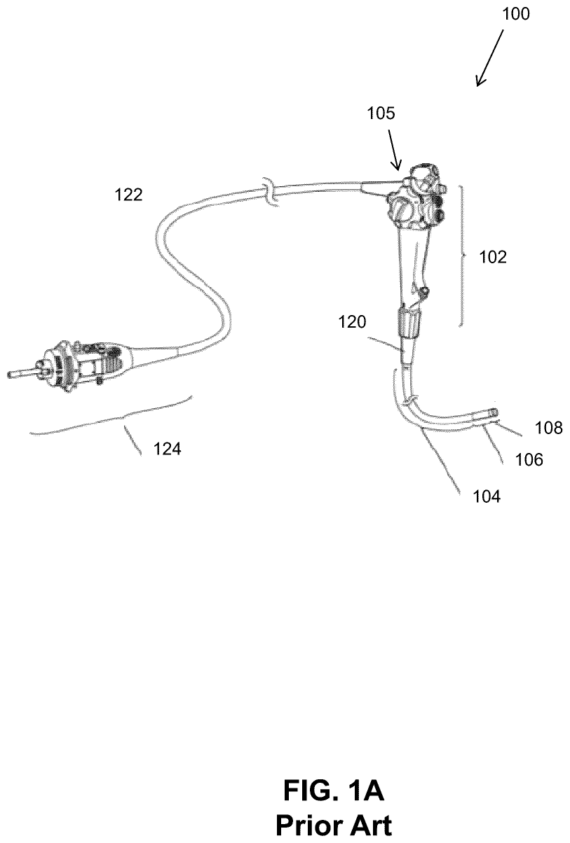

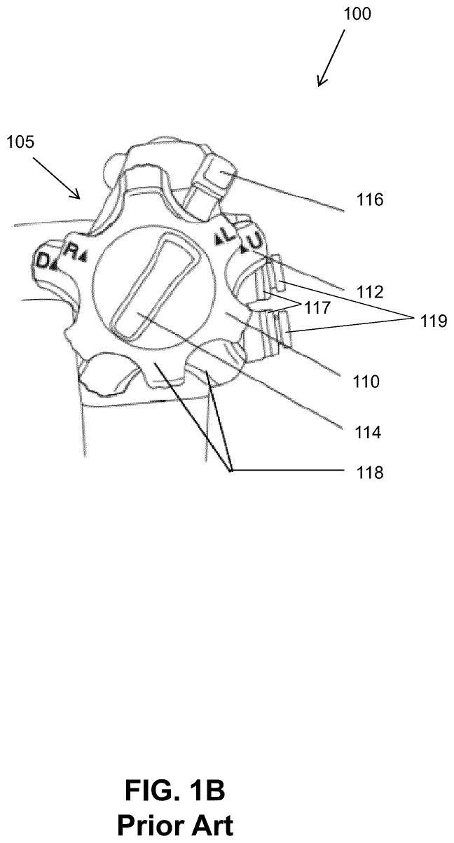

FIGS. 1A and 1B are perspective views of an endoscope device 100 as known in the prior art. The endoscope device includes a body 102 and an insertion tube 104. The body 102 includes a control system 105 including a plurality of knobs to control movement of a bendable tube segment 106 at a distal end 108 of the insertion tube 104, including a first angulation control knob 110 to control right/left movement and a second angulation control knob 112 to control up/down movement. The body 102 also includes a first lock 114 and a second lock 116 to lock the first and second angulation control knobs 110, 112, respectively, in place, thereby locking the position of the bendable tube segment 106 of the insertion tube 104. The first and second angulation control knobs 110, 112 each include a plurality of knob protrusions 118. The body 102 further includes one or more valve cylinders 117 for insertion of one or more valves 119, such as, but not limited to, a suction valve and an air/water valve.

The endoscope device 100 includes a boot 120 that couples the body 102 to the insertion tube 104. Additionally, the endoscope device 100 includes an umbilicus 122 and a connector 124 to connect the endoscope device 100 to a power source. A distal end of the umbilicus 122 is coupled to the body 102 and a proximal end of the umbilicus is coupled to the connector 124. The umbilicus may be coupled to the body 102 and/or the connector 124 by a boot substantially similar to the boot 120. The connector 124 may connect the endoscope device 100 to a tower that includes a computer system, a display, and/or entry ports for air and water intake.

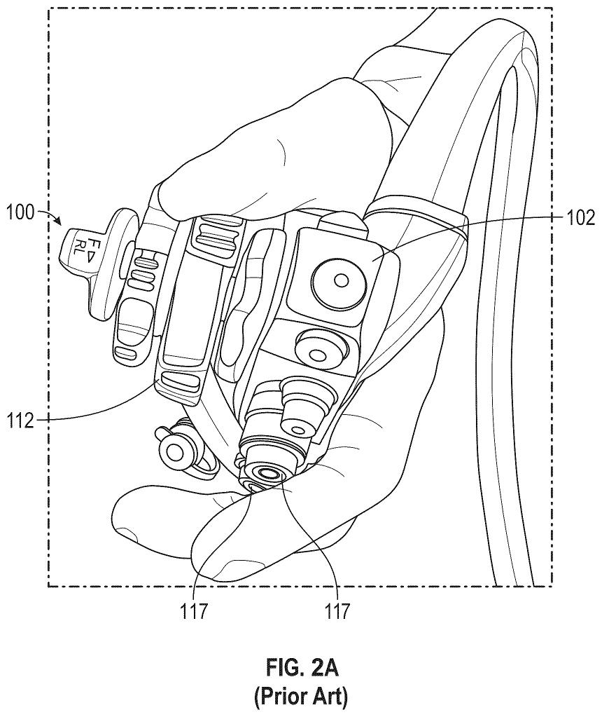

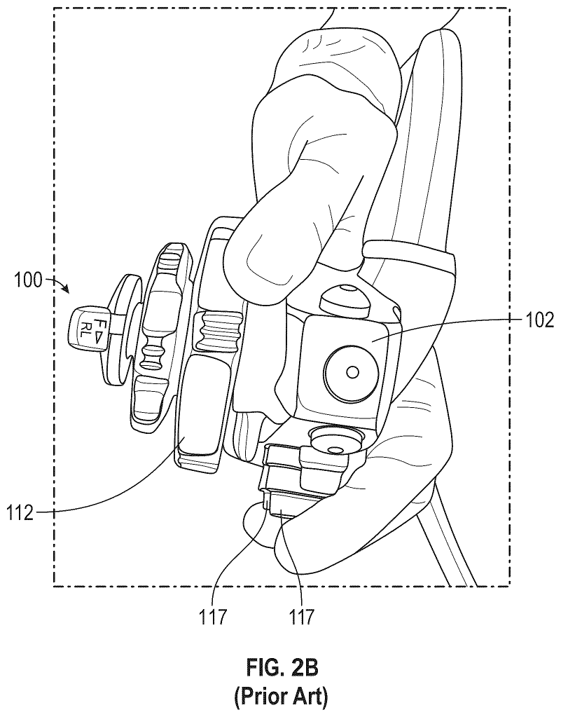

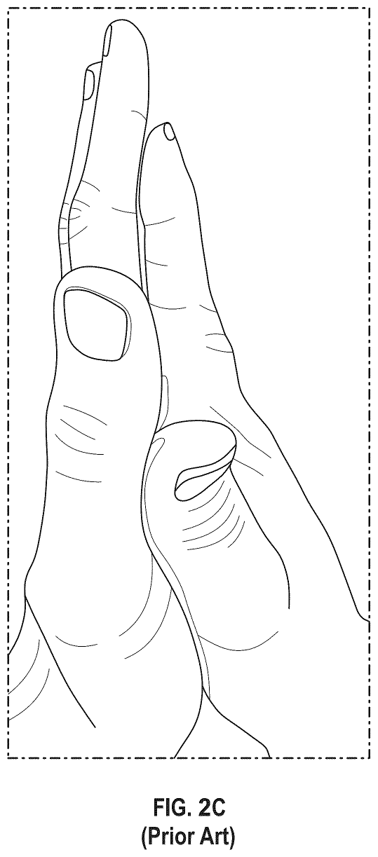

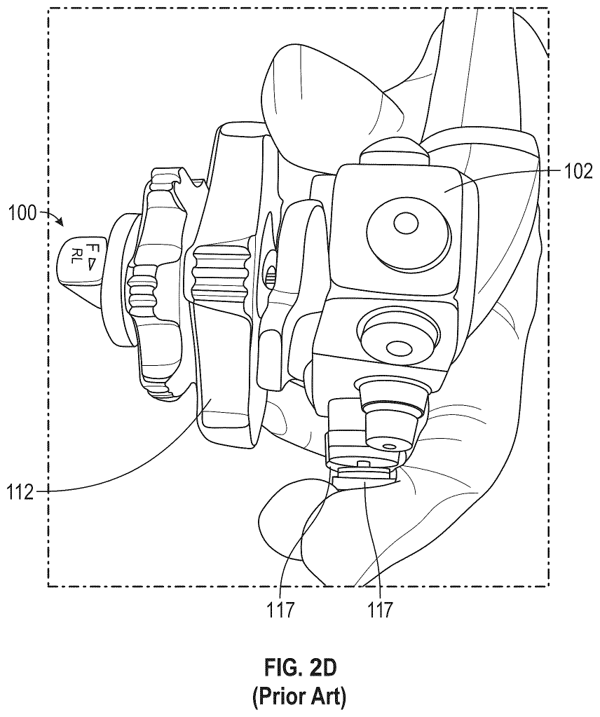

FIGS. 2A and 2B are perspective views of an operator using the endoscope device 100. As shown in FIG. 2A, a resting grip of a hand of an operator may include a thumb on the second angulation control knob 112 and an index finger and/or a middle finger extending past the one or more valve cylinders 117. As shown in FIG. 2B, an active grip of a hand of an operator to use the endoscope device 100 may include the thumb on the second angulation control knob 112 and the index finger and/or the middle finger in a semi-clawed position on the one or more valve cylinders 117. The semi-clawed position of the fingers in the active grip may require the operator to exert high pinch forces, endure contact stress, and/or maintain awkward postures while operating the endoscope device 100. This may increase the operator's risk of sustaining work-related musculoskeletal injuries.

FIG. 2C is a side view illustrating an example difference in hand size. FIG. 2D is a perspective view of an operator with a smaller hand size using the endoscope device 100. As shown in FIG. 2D, an active grip of a hand of an operator with a smaller hand size to use the endoscope device 100 may include the thumb on the body 102 and the index finger and/or the middle finger in a minimal semi-clawed position on the one or more valve cylinders 117. The required reach of the thumb to move to the first angulation control knob 110 and/or the second angulation control knob 112 while keeping the minimal semi-clawed position of the index and middle fingers may increase the operator's risk of sustaining work-related musculoskeletal injuries.

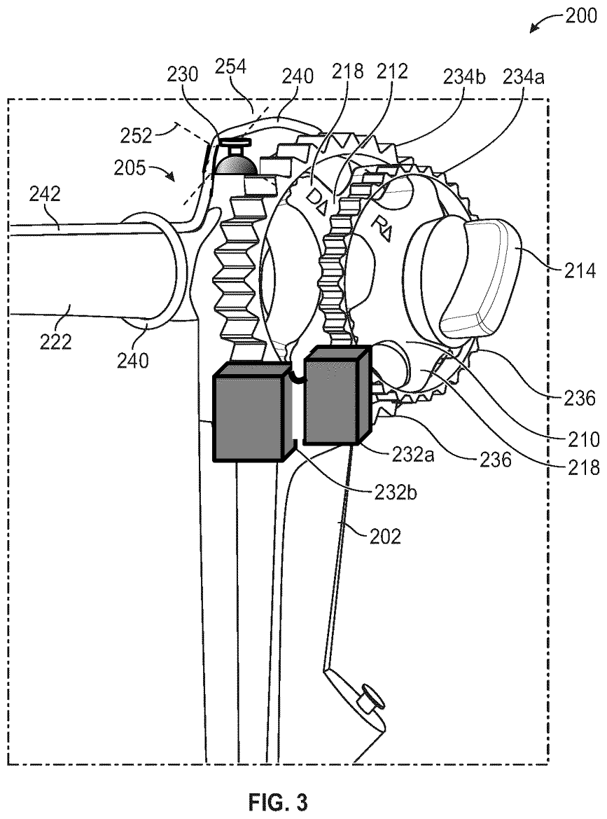

FIG. 3 is a schematic of a perspective view of an endoscope device 200. The endoscope device 200 shares similarities with the previously described embodiment of the endoscope device 100. As such, certain components or parts of the endoscope device 200 may correspond to or be substantially similar to those described for the endoscope device 100. Accordingly, where applicable, similar or identical part numbers may be used to denote corresponding components.

The endoscope device 200 includes a body 202 and an insertion tube (not shown). The body 202 includes an auxiliary control system 205 including a plurality of knobs to control movement of a bendable tube segment (not shown) at a distal end (not shown) of the insertion tube (not shown), including a first angulation control knob 210 to control right/left movement and a second angulation control knob 212 to control up/down movement. The body 202 also includes a first lock 214 and a second lock (not shown) to lock the first and second angulation control knobs 210, 212, respectively, in place, thereby locking the position of the bendable tube segment (not shown) of the insertion tube (not shown). The first and second angulation control knobs 210, 212 each include a plurality of knob protrusions 218. In some embodiments, the knob protrusions 218 may be substantially similar for both the first and second angulation control knobs 210, 212. In other embodiments, the knob protrusions 218 may be different between the first and second angulation control knobs 210, 212, such as, but not limited to, in number, shape, length, and/or width.

The body 202 further includes one or more valve cylinders (not shown) for insertion of one or more valves (not shown), such as, but not limited to, a suction valve and an air/water valve. The endoscope device 200 also includes an umbilicus 222 and a connector (not shown) to connect the endoscope device 200 to a power source. A distal end of the umbilicus 222 is coupled to the body 202 and a proximal end of the umbilicus is coupled to the connector (not shown).

In one aspect, as shown, the auxiliary control system 205 includes a joystick 230, a plurality of knob motors 232, and a plurality of knob engagement discs 234. The auxiliary control system 205 differs from the control system 105 in that the joystick 230 may be used to control movement of the first and second angulation control knobs 210, 212 via the knob motors 232 and the knob engagement discs 234, the joystick 230 being positioned for improved ergonomic operation of the endoscope device. Specifically, the joystick 230 is positioned on the body 202 proximate to the position of a thumb of a hand of an operator in an active grip.

The knob engagement discs 234 are sized to fit around the first and second angulation control knobs 210, 212. That is, a radius of the knob engagement discs 234 is sized to be slightly larger than a radius of the corresponding angulation control knob as measured at the knob protrusions 218. Accordingly, when the knob engagement discs 234 are moved, the corresponding angulation control knob is likewise moved due to the engagement of the knob engagement discs 234 with the knob protrusions 218 of the angulation control knobs 210, 212.

As shown in FIG. 3, a first knob motor 232a is positioned on the body 202 proximate the first angulation control knob 210 and a second knob motor 232b is positioned on the body 202 proximate the second angulation control knob 212. The knob motors 232 are geared and positioned such that the gears engage with disc protrusions 236 around the circumferential surface of the knob engagement discs 234. When activated, the gears of the first knob motor 232a engage with the disc protrusions 236 of a first knob engagement disc 234a to move the first knob engagement disc 234a and thereby the first angulation control knob 210. Additionally, when activated, the gears of the second knob motor 232b engage with the disc protrusions 236 of a second knob engagement disc 234b to move the second knob engagement disc 234b and thereby the second angulation control knob 212.

The knob engagement discs 234 may be designed for use with existing endoscope devices. That is, the knob engagement discs 234 may be attachable and detachable from angulation control knobs of existing endoscope devices known in the prior art. In some embodiments, the knob engagement discs 234 may be of any elastic material, such as, but not limited to, rubber or polymer. The elasticity may allow the knob engagement discs 234 to be stretched over the corresponding angulation control knob for a secure fit. In other embodiments, the knob engagement discs 234 may snap on to the corresponding angulation control knob for a secure fit. For example, the knob engagement discs 234 may latch underneath the corresponding angulation control knob to keep the knob engagement discs 234 in place. The knob engagement discs 234 may be designed to be of a minimal weight for ease of attachment and portability.

The knob engagement discs 234 may include one or more inner protrusions on an inner circumferential surface to engage with the one or more knob protrusions 218 of the corresponding angulation control knob. The number of inner protrusions of the knob engagement discs 234 may depend on the number of knob protrusions 218 on the corresponding angulation control knob. For example, the first knob engagement disc 234a may include up to six inner protrusions and the second knob engagement disc 234b may include up to five inner protrusions. The inner protrusions may be shaped to securely fit around one of the knob protrusions 218. The number of inner protrusions of the knob engagement discs 234 may be the same or may be different than the number of knob protrusions 218 on the corresponding angulation control knob.

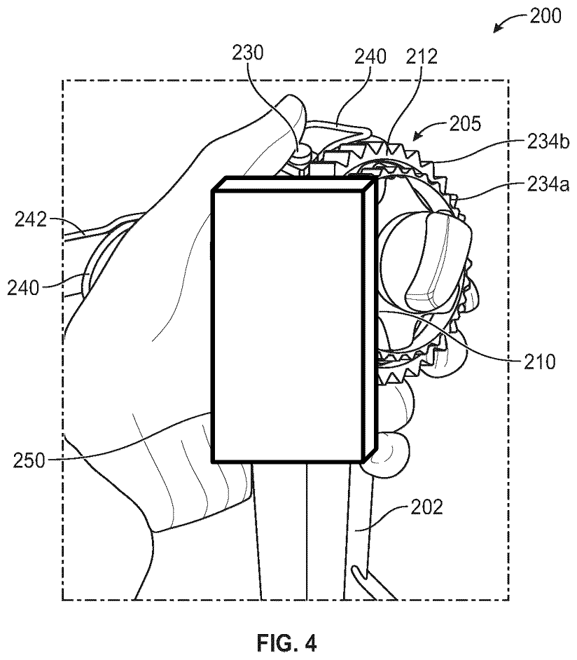

FIG. 4 is a schematic of a perspective view of the endoscope device 200 including the auxiliary control system 205. As shown in FIG. 4, the auxiliary control system 205 may include a panel 250 to cover at least a portion of the auxiliary control system 205. The panel 250 may be positioned to protect the hand of an operator of the endoscope device 200 from the knob motors 232. That is, the panel 250 may cover the knob motors 232 to allow the operator to continue using the auxiliary control system 205 while the knob motors 232 remain proximate to the corresponding angulation control knob. The auxiliary control system 205 may also include a switch (not shown) to stop unintentional motor activity of the knob motors 232 and allow the operator to continue use of the endoscope device 200. Positioning of the panel 250 on the body 202 may require repositioning of the knob motors 232 on the body 202. For example, the position of the knob motors 232 below the corresponding angulation control knob may be different than that shown in FIG. 3, such as at a slight offset so the knob motors 232 are not directly beneath the corresponding angulation control knob. Additionally, for example, the knob motors 232 may be repositioned to be oriented at an angle relative to the body 202.

When operating the auxiliary control system 205, an operator may move the joystick 230 along a first axis 252 to activate the first knob motor 232a and move the first knob engagement disc 234a and thereby the first angulation control knob 210. Additionally, the operator may move the joystick along a second axis 254 perpendicular to the first axis to activate the second knob motor 232b and move the second knob engagement disc 234b and thereby the second angulation control knob 212. For example, moving the joystick 230 left or right along the first axis 252 may spin the first angulation control knob 210 left or right, respectively, and deflect the bendable tube segment (not shown) at the distal end (not shown) of the insertion tube (not shown) in a left or right direction, respectively. Additionally, for example, moving the joystick 230 up or down along the second axis 254 may spin the second angulation control knob 212 forward or backward, respectively, and deflect the bendable tube segment (not shown) at the distal end (not shown) of the insertion tube (not shown) in a down or up direction, respectively.

The auxiliary control system 205 may also include an inversion switch (not shown) to invert the relationship between the second axis 254 and the spin direction of the angulation control knob 212. For example, the inversion switch (not shown) may allow the joystick 230 to be moved up or down along the second axis 254 to spin the second angulation control knob 212 backward or forward, respectively, and deflect the bendable tube segment (not shown) at the distal end (not shown) of the insertion tube (not shown) in an up or down direction, respectively.

When operating the auxiliary control system 205, an operator may move the joystick 230 along both the first axis 252 and the second axis 254 concurrently. That is, the operator may move the joystick up or down along the second axis 254 and right or left along the first axis 252 such that the joystick is moved diagonally to deflect the bendable tube segment (not shown) at the distal end (not shown) of the insertion tube (not shown) in two directions at once.

In some embodiments, the operator may click the joystick 230 to return the first and second angulation control knobs 210, 212 to their original position, thereby resetting the position of the bendable tube segment (not shown) at the distal end (not shown) of the insertion tube (not shown). For example, clicking the joystick 230 may reset the position of the bendable tube segment (not shown) to be straightened out (e.g., not bent in a left/right or an up/down direction). In other embodiments, the operator may click the joystick 230 to initiate custom controls of the joystick 230. For example, clicking the joystick 230 may position the bendable tube segment (not shown) of the insertion tube (not shown) in a specific position as an alternative locking feature to the angulation locks. Additionally, for example, clicking the joystick 230 may change the control axes of the joystick 230 such that movement along the first axis 252 activates the second knob motor 232b to move the second angulation control knob 212 and movement along the second axis 254 activates the first knob motor 232a to move the first angulation control knob 210.

In some embodiments, the tilt magnitude of the joystick 230 may correlate to the spin speed of the first and second angulation control knobs 210, 212. For example, the greater the tilt magnitude of the joystick 230, the faster the resulting spin speed of the first and second angulation control knobs 210, 212 and the faster the deflection of the bendable tube segment (not shown) at the distal end (not shown) of the insertion tube (not shown).

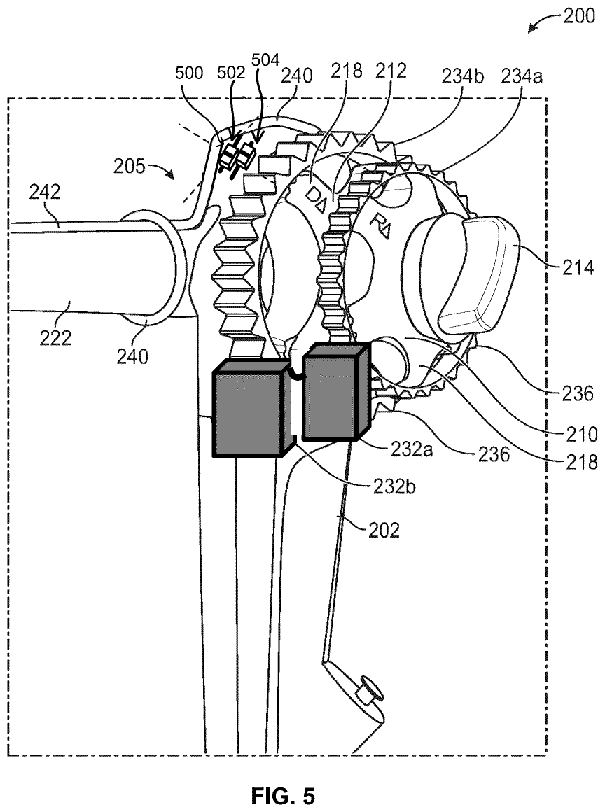

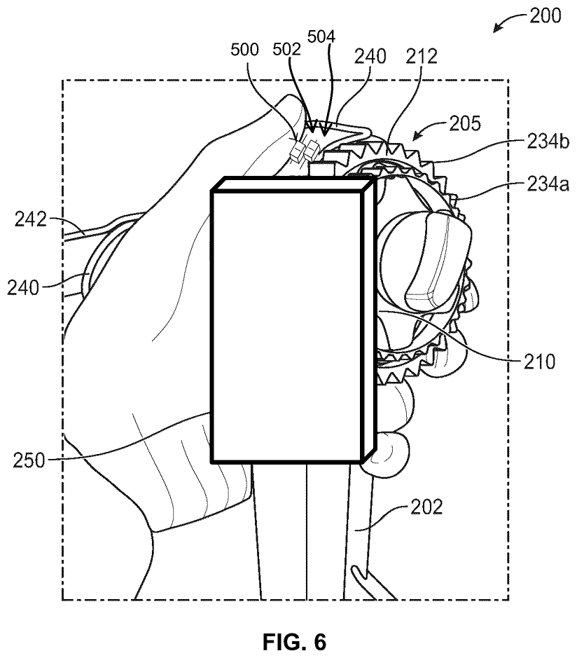

Referring now to FIGS. 5 and 6, another embodiment of the auxiliary control system 205 referred to herein as a slider system includes a slider module 500 having one or more sliders. In one aspect, the sliding module 500 includes a one or more slider, a plurality of geared motors and a cover plate. In one aspect, the plurality of geared motors and cover plate are similar to those as previously described with reference to FIGS. 3 and 4.

In various aspects, the sliding module 500 includes a dual slider configuration wherein one slider 502 controls U/D movements and the other slider 504 controls L/R movements. According to various aspects, the sliders are configured such that moving the first slider 502 up and down would cause the first motor 232A to rotate the U/D control knob 210 forward and backward, respectively. Sliding the second slider 504 up and down causes the second motor 232B to spin the L/R control knob 212 forward and backward, respectively.

As shown in FIG. 5, the first motor 232B interfaces with U/D engagement disk 234B and the second motor 232A interfaces with the L/R engagement disk 234A. As shown, in one aspect, the motors communicate with the slider module 500 that is positioned proximal to the U/D angulation dial, typically where the left thumb would naturally rest.

The cover panel 250 serves as a safety barrier between the users and motors and gears. The cover panel 250 is also configured to secure the motors and ensure that the motors remain tangent to the geared engagement disks 234A-B at all times.

According to at least one aspect, sliding the first slider 502 upward leads to U/D motor spinning the engagement disked U/D dial forward, deflecting endoscope tip downward and sliding the first slider 502 downward leads to U/D motor spinning the engagement disked U/D dial backward, deflecting endoscope tip upward. Similarly, sliding second slider 504 upward leads to L/R motor spinning the engagement disked L/R dial forward, deflecting endoscope tip rightward, while sliding second slider downward leads to L/R motor spinning the engagement disked L/R dial backward, deflecting endoscope tip leftward

According to various other aspects, the slider module 500 includes indica to provide the user with an indication of the degree of deflection. As such, the user can gauge more precisely the actual deflection of the endoscope tip. According to one aspect, there is a direct relationship between slider displacement and endoscope tip deflection. For example, moving the slider three-fourths (¾) of the travel distance in the Up direction of the U/D slider, may cause the endoscope tip to deflect three-fourths (¾) of the maximum possible travel distance in the Up direction. According to one aspect, the maximum travel distance of the endoscope may be limited by the user. As such, moving the slider three-fourths (¾) of the travel distance in the Up direction of the U/D slider, may cause the endoscope tip deflect three-fourths (¾) of the limited maximum possible travel distance in the Up direction.

In yet other aspects, the speed at which the endoscope tip deflects may be directly related to the speed and/or magnitude of the slider translation. For example, the greater the magnitude of slider tilt relative to the original position in one aspect, or relative to a current position in another aspect, then the greater the motor speed, speed of engagement disk spin, and speed of endoscope tip deflection. As such, a user can quickly deflect the tip of the endoscope. Conversely, with smaller magnitudes of slider tilt relative to the original position in one aspect, or relative to the current position in another aspect, the slower the motor and engagement disk will spin, resulting in a slower speed of endoscope tip deflection.

According to one or more aspects, depressing the sliders, regardless of their current position will cause the motors to return to their original non-deflected position or neutral positions, thus resetting the flexible section of the insertion tube to its native position. Furthermore, the sliders will also return their original or neutral positions upon depressing the sliders.

In various alternative aspects, the assumed position to which the endoscope returns upon depressing the sliders may not be the original non-deflected position, but rather to a custom configuration that may be configured during a procedure or may be a pre-determined default position as determined by the user. For example, depressing the slider may cause the endoscope to assume a particular deflection that has been pre-determined as the optimal position for entering or withdrawing from a biological structure or conduit.

In various aspects, the slider module 500 may also include an inversion switch (not shown) to invert the relationship between the corresponding axis and the spin direction of the angulation control knob, similar to that described with the joystick-based auxiliary control system 205.

In various aspects, the slider module 500 can be secured onto the control section of the endoscope using a latch, hook and loop fasteners or any other suitable means. Power may be provided to the slider module 500 via a USB cable connected to a computing device.

According to various aspects, the slider module 500 is detachable from the endoscope and portions thereof may be reused for subsequent procedures. For example, once a procedure is completed, the slider module 500 and the dial engagement disks 234A-B can be detached from the endoscope. The slider module 500 can be manually wiped down, sprayed with a disinfectant or cleaned and disinfected by any other suitable means. Moreover, the dial engagement disks may be configured as a consumable product and therefore disposed after use. Alternatively, the dial engagement disks may be composed of a resilient material that is capable of efficient cleaning or disinfection.

Elements of the auxiliary control system 205 may be removably attachable to an endoscope device known in the prior art, such as, but not limited to, the endoscope device 200. For example, the knob motors 232 may be secured to the body of the endoscope device, such as the body 202, by a latch (such as latches 240 shown in FIG. 3) or a hook and loop attachment mechanism. Additionally, for example, the knob engagement discs 234 may be secured to the corresponding angulation control knob by a snap mechanism. The auxiliary control system 205 may include a cord 242 with a USB plug (not shown) to connect to a power source. The cord (not shown) may be a substantially similar length as the umbilicus 222 (shown in FIG. 3) and may be secured to the umbilicus 222 by a hook and loop attachment mechanism.

According to various aspects, the present disclosure also relates to knob motors 232 that may be affixed to or are integrated with the endoscope device, wherein the knob motors may operate independently of the auxiliary control system 205. Moreover, in some aspects, the endoscope device includes internal knob motors (not shown) that can rotate the control knobs 210 and 212 directly without requiring engagement discs 234. In these aspects, the auxiliary control system 205 is optional and the knob motors 232 may provide assistance to the user's manual rotation of the control knobs similar to that provided by the pedal-assist functionality of an electro bicycle. Furthermore, the knob motors 232 are configured to provide assistance that is proportional to the user's effort, which may reduce user fatigue during lengthy procedures.

The disclosures shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the examples described above may be modified within the scope of the appended claims.

What is claimed is:

1. An endoscope system comprising:
an insertion tube comprising a bendable tube segment at a distal end of the insertion tube;
a body comprising a plurality of angulation control knobs configured to control positioning of the bendable tube segment of the insertion tube;
a control system comprising an input device, a knob motor positioned proximate to each angulation control knob, and a knob engagement disc releasably secured to each angulation control knob;
wherein the input device is communicatively coupled to each knob motor such that movement of the input device activates the knob motors and controls movement of the angulation control knobs via the knob engagement discs; and
wherein positioning of the input device facilitates improving one or more ergonomic conditions of a hand grip of an operator of the endoscope system.

2. The endoscope system of claim 1, wherein the plurality of angulation control knobs comprise a first angulation control knob configured to control positioning along a first bending axis and a second angulation control knob configured to control positioning along a second bending axis.

3. The endoscope system of claim 2, wherein a first knob motor is positioned proximate to the first angulation control knob and a second knob motor is positioned proximate to the second angulation control knob.

4. The endoscope system of claim 2, wherein the input device is a joystick and wherein the when the joystick is moved along a first axis to control positioning of the bendable tube segment of the insertion tube along the first bending axis and the joystick is moved along a second axis to control positioning of the bendable tube segment of the insertion tube along the second bending axis.

5. The endoscope system of claim 4, wherein the first axis is substantially parallel to the first bending axis in a left-right orientation and the second axis is substantially parallel to the second bending axis in an up-down orientation.

6. The endoscope system of claim 1, wherein the knob motors comprise one or more gears to engage with one or more protrusions around an exterior circumferential surface of one of the knob engagement discs.

7. The endoscope system of claim 1, wherein the input device comprises a slider, wherein moving the slider along an axis in a first direction causes the bendable tube segment of the insertion tube to bend along a bending axis in the first direction and wherein moving the slider in a second direction along the axis causes the bendable tube segment of the insertion tube to bend along the first bending axis opposite the first direction.

8. The endoscope system of claim 7, wherein the input device comprises another slider and wherein moving the other slider along in the first direction causes the bendable tube segment of the insertion tube to bend along a second bending axis in a second direction and wherein moving the slider in the second direction along the axis causes the bendable tube segment of the insertion tube to bend along the second bending axis opposite the first direction.

9. The endoscope system of claim 1, wherein the input device comprises a slider and wherein a speed of the slider along an axis in a first direction correlates to a speed of deflection for the bendable tube segment.

10. The endoscope system of claim 1, wherein the input device comprises a slider and wherein a distance of the slider from a neutral position corresponds to a of deflection for the bendable tube segment.

11. The endoscope system of claim 1, wherein the input device comprises a slider and wherein depressing the slider causes the bendable tube segment to return to a neutral position.

12. The endoscope system of claim 1, wherein the input device comprises two sliders and wherein depressing a first slider causes the bendable tube segment to return to a neutral position along a first axis and depressing a second slider causes the bendable tube segment to return to a neutral position along a second axis.

13. The endoscope system of claim 1, wherein the input device and the knob motors are releasably secured to the body.

14. The endoscope system of claim 1, wherein the body includes a panel to cover at least a portion of the control system.

15. The endoscope system of claim 14, wherein the panel covers the knob motors.

16. The endoscope system of claim 1, wherein the control system is detachable from the body and the insertion tube.

17. The endoscope system of claim 1, wherein the control system is integrated with the body.

18. A method of using an endoscope system, the endoscope system comprising an insertion tube comprising a bendable tube segment at a distal end of the insertion tube, a body comprising a plurality of angulation control knobs configured to control positioning of the bendable tube segment of the insertion tube, and a control system comprising a, input device, a knob motor positioned proximate to each angulation control knob, and a knob engagement disc releasably secured to each angulation control knob, wherein the input device is communicatively coupled to each knob motor such that movement of the input device activates the knob motors and controls movement of the angulation control knobs via the knob engagement discs, the method comprising:
inserting the distal end of the insertion tube into a patient;
advancing the distal end of the insertion tube to a desired location within the patient;
operating the endoscope system, wherein positioning of the input device facilitates improving one or more ergonomic conditions of a hand grip of an operator of the endoscope system; and
removing the insertion tube from the patient.

19. The method of claim 18, wherein the plurality of angulation control knobs comprise a first angulation control knob configured to control positioning along a first bending axis and a second angulation control knob configured to control positioning along a second bending axis, and wherein a first knob motor is positioned proximate to the first angulation control knob and a second knob motor is positioned proximate to the second angulation control knob.

20. The method of claim 18, wherein the input device is a joystick and when the joystick is moved along a first axis to control positioning of the bendable tube segment of the insertion tube along a first bending axis and the joystick is moved along a second axis to control positioning of the bendable tube segment of the insertion tube along the second bending axis.

21. The method of claim 18, wherein the input device comprises one or more sliders and wherein when a first slider is moved along a first axis to control positioning of the bendable tube segment of the insertion tube along a first bending axis and a second slider is moved along a second axis to control positioning of the bendable tube segment of the insertion tube along a second bending axis.

22. The method of claim 21, wherein depressing the first slider causes the bendable tube segment of the insertion tube to return to a neutral position along the first bending axis or depressing the second slider causes the bendable tube segment of the insertion tube to return to the neutral position along the second bending axis.

23. The method of claim 18, wherein the knob motors comprise one or more gears to engage with one or more protrusions around an exterior circumferential surface of one of the knob engagement discs.

24. The method of claim 18, wherein the one or more ergonomic conditions comprise at least one of a force exertion, a contact stress, and a posture.

25. The method of claim 24, wherein the hand grip of the operator includes a thumb positioned to move the input device.
